(19) United States
(12) Patent Application Publication
(10) Pub. No.: US 2026/0325451 A1
(43) Pub. Date: Oct. 22, 2026

(54) PROGRAMMABLE DELAYED-RELEASE CONTAINER SYSTEM FOR ORAL DRUG DELIVERY

(71) Applicants: Ahmed Wadi, West Roxbury, MA (US); Mohammed Maniruzzaman, Oxford, MS (US)

(72) Inventors: Ahmed Wadi, West Roxbury, MA (US); Mohammed Maniruzzaman, Oxford, MS (US)

(21) Appl. No.: 19/652,796

(22) Filed: Apr. 20, 2026

Related U.S. Application Data
(60) Provisional application No. 63/792,699, filed on Apr. 22, 2025.

(51) Int. Cl. A61K 9/48; A61K 9/00

(52) U.S. Cl. CPC A61K 9/4808; A61K 9/0056

(57) ABSTRACT

A delayed-release oral dosage form comprising a container structure defining an internal cavity, a swellable composition disposed within the internal cavity, and an active pharmaceutical ingredient. The container structure includes one or more ingress openings configured to permit entry of external fluid into the internal cavity while substantially preventing release of the active pharmaceutical ingredient prior to rupture of the container structure. Upon exposure to fluid, the swellable composition hydrates and expands within the confined cavity, generating internal pressure. The pressure increases until rupture of at least a portion of the container structure occurs, thereby releasing the active pharmaceutical ingredient after a delay period following administration.

### CROSS-REFERENCE TO RELATED APPLICATIONS

This application claims priority to U.S. Provisional Patent Application No. 63/792,699, filed on Apr. 22, 2025, entitled “Time Programmable Delayed Release Container System,” the entire contents of which are incorporated herein by reference.

### FIELD OF THE INVENTION

The present invention relates generally to pharmaceutical drug delivery systems and, more particularly, to oral dosage forms capable of providing programmable delayed release of an active pharmaceutical ingredient through pressure-induced rupture of a container structure.

### BACKGROUND OF THE INVENTION

Delayed-release drug delivery systems have been developed to control the timing of drug release following administration. Such systems are particularly useful when therapeutic effectiveness depends on synchronization of drug availability with physiological or pathological processes, including circadian biological rhythms.

A variety of technologies have been explored to achieve delayed release in oral dosage forms. One widely-used strategy involves coating-based systems, in which the drug-containing core is surrounded by a polymeric coating that delays drug release until the coating dissolves, erodes, or ruptures. Examples include enteric coatings designed to dissolve at elevated intestinal pH and time-dependent coatings that gradually erode or become permeable during gastrointestinal transit.

Other approaches include press-coated tablets and capsule plug systems, in which a barrier layer or plug delays exposure of the drug-containing core until the barrier erodes or is expelled. Multiparticulate systems, including coated pellets or beads, have also been used to generate delayed or pulsatile release profiles by applying functional coatings to individual particles.

In addition, osmotic delivery devices have been developed in which fluid enters the dosage form through a semipermeable membrane, generating osmotic pressure that drives drug release through a delivery orifice. While osmotic systems are primarily designed for controlled release, certain variations have been adapted to produce delayed or pulsatile release profiles.

Other delayed-release strategies include matrix systems, hydrogel-based systems, erodible barrier systems, and geometry-controlled devices in which release timing depends on polymer swelling, erosion, or diffusion processes occurring within the dosage form.

Despite the diversity of these approaches, most existing systems rely on polymer coatings, membrane permeability, erosion, or diffusion-controlled mechanisms to regulate drug release.

Delayed-release oral dosage forms have been widely investigated as a strategy for controlling the timing of drug release following administration. Such systems are of particular interest in chronotherapeutic applications where drug release after a defined lag period may improve therapeutic outcomes. Various approaches have been developed to achieve delayed drug release, including coating-based systems, osmotic delivery devices, multiparticulate dosage forms, and multilayer tablet architectures.

In coating-based systems, the lag time preceding drug release may be influenced by parameters such as coating thickness, membrane permeability, and polymer dissolution or erosion behavior. Achieving consistent lag times in such systems may depend on maintaining uniform coating characteristics and appropriate material properties during manufacturing and storage.

Other delayed-release technologies employ multiparticulate systems, multilayer tablet structures, or osmotic pumping mechanisms to regulate drug release after a predetermined delay period. In these systems, the timing of release may be governed by interactions between formulation composition, structural design, and environmental conditions within the gastrointestinal tract.

Although these approaches have enabled delayed-release behavior in a variety of pharmaceutical formulations, achieving precise and programmable control over the lag period preceding drug release remains a challenge in the design of oral drug delivery systems. Many existing technologies regulate delayed release through combinations of membrane permeability, polymer dissolution, coating thickness, osmotic transport, or related physicochemical processes.

Accordingly, a remaining need exists for the development of delayed-release oral dosage forms capable of providing programmable lag times through alternative dosage form architectures and design approaches.

Hydrocortisone replacement therapy for Addison's disease provides a representative example of a therapeutic situation in which delayed and programmable oral drug release may be especially valuable. Addison's disease, or primary adrenal insufficiency, is characterized by impaired adrenal hormone production and is commonly managed by oral hydrocortisone administration intended to mimic the circadian rhythm of endogenous cortisol secretion. Endogenous cortisol normally follows a pronounced temporal profile, with levels rising to an early-morning peak and declining thereafter during the day. Conventional hydrocortisone regimens therefore attempt to reproduce this physiological rhythm through multiple daily doses. However, standard schedules may not adequately account for inter-individual differences in absorption, metabolism, daily routine, work schedule, age, pregnancy, or physiological stress, all of which may influence the timing and magnitude of cortisol requirements. Accordingly, there remains a need for oral dosage forms capable of providing programmable delay periods that can be adjusted through dosage form design in order to better align hydrocortisone release with patient-specific therapeutic needs.

Other disease states may also benefit from oral dosage forms capable of programmable delayed release. Bronchial asthma is a well-recognized chronotherapeutic target because airway resistance and symptom exacerbation often worsen at night or in the early morning; accordingly, oral bronchodilators or anti-inflammatory drugs such as theophylline may benefit from timed delivery aligned with these periods of increased need. Rheumatoid arthritis is another example in which pain, stiffness, and inflammatory activity frequently peak in the morning, making oral anti-inflammatory agents such as diclofenac or indomethacin suitable candidates for delayed-release administration timed to anticipate symptom onset. Cardiovascular disorders likewise exhibit circadian patterns, with increased incidence of events such as myocardial ischemia, angina, arrhythmias, and myocardial infarction during the morning hours; oral cardiovascular drugs such as verapamil, propranolol, or nitrates may therefore benefit from programmed delivery profiles. Similar considerations have been described for diabetes, where circadian variation in insulin requirement and glucose regulation may support time-specific administration of oral antidiabetic agents, and for hypercholesterolemia, in which circadian variation in cholesterol synthesis has motivated timed administration of orally delivered HMG-CoA reductase inhibitors. These examples illustrate the broader therapeutic relevance and need for oral delivery systems capable of providing programmable lag times prior to drug release.

### SUMMARY OF THE INVENTION

The present invention provides a delayed-release oral composition capable of producing a programmable lag period prior to release of at least one active pharmaceutical ingredient. The composition comprises a structurally defined container architecture in which fluid ingress is spatially restricted and internal pressure accumulation governs the timing of release of the at least one active pharmaceutical ingredient.

In one aspect, the invention provides a delayed-release oral composition comprising a container structure defining an internal cavity. A swellable composition is contained within the internal cavity, and an active pharmaceutical ingredient is positioned within the container structure. One or more predefined ingress openings extend through at least one wall (e.g., top, bottom, and/or lateral surface) of the container structure, for example as illustrated in FIG. 1.

The container structure is substantially impermeable to gastrointestinal fluid except through the predefined ingress openings. Gastrointestinal fluid enters the internal cavity through the predefined ingress openings, where it contacts the swellable composition. Hydration of the swellable composition results in volumetric expansion within the internal cavity, generating internal pressure against the surrounding container structure.

The predefined ingress openings are configured to permit controlled entry of gastrointestinal fluid while preventing release of the active pharmaceutical ingredient prior to rupture of the container structure. As hydration proceeds, internal pressure increases until the internal pressure exceeds the mechanical strength of the container structure. When this condition is reached, structural rupture of the container structure occurs, thereby releasing the active pharmaceutical ingredient after a controlled delay period, as illustrated in FIGS. 2A-2C.

In certain embodiments, the delay period preceding rupture is governed at least in part by geometric parameters of the predefined ingress openings. Such parameters may include the size, number, spatial distribution, and cross-sectional geometry of the ingress openings.

In certain embodiments, the active pharmaceutical ingredient is arranged in a layer separate from the swellable composition. An optional separating layer may be positioned between the swellable composition and the active pharmaceutical ingredient.

In some embodiments, the container structure defines a single internal cavity in which hydration and pressure accumulation occur. In other embodiments, the container structure defines multiple internal compartments configured to contain different compositions or therapeutic agents, enabling staged or multi-phase release profiles.

The container structure can assume a variety of geometries including cylindrical, spherical, ellipsoidal, polygonal, or other suitable shapes. The container structure can be formed from polymeric materials capable of maintaining structural integrity during the lag phase prior to rupture.

The dosage form can be manufactured using pharmaceutical processing techniques including additive manufacturing, extrusion-based fabrication, molding, casting, or other suitable fabrication methods.

The present invention therefore provides a delayed-release oral dosage form in which fluid ingress is restricted to defined structural pathways and release is triggered by pressure-induced rupture within a confined structure, enabling programmable control of the delay period prior to drug release.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 depicts a cross-sectional view of an embodiment of the delayed-release container system showing the container shell, internal cavity, swellable composition, separating layer, active pharmaceutical ingredient layer, and ingress openings.

FIG. 2A depicts an embodiment of the delayed-release container system in an intact state prior to ingress of gastrointestinal fluid.

FIG. 2B depicts the delayed-release container system during operation, showing hydration and expansion of the swellable composition following ingress of gastrointestinal fluid through the ingress openings.

FIG. 2C depicts rupture of the container structure following pressure generation within the internal cavity and subsequent release of the active pharmaceutical ingredient.

FIG. 3A depicts an embodiment of the delayed-release container system comprising relatively small ingress openings.

FIG. 3B depicts an embodiment of the delayed-release container system comprising medium-sized ingress openings.

FIG. 3C depicts an embodiment of the delayed-release container system comprising relatively large ingress openings.

FIG. 4 depicts a representative drug release profile showing the lag-time behavior of the delayed-release container system.

FIG. 5 depicts the swelling behavior of the swellable composition, hydroxypropyl methylcellulose, in different pH representing the gastrointestinal tract.

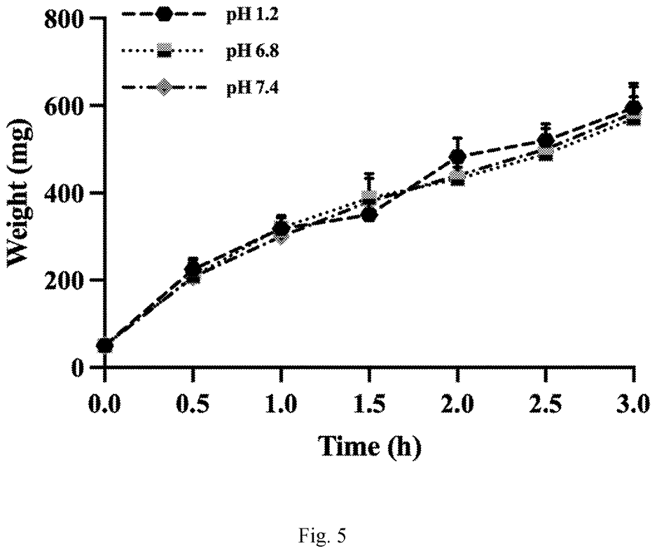

### DETAILED DESCRIPTION

The present disclosure relates to delayed-release pharmaceutical delivery systems and, more specifically, to a programmable delayed release container system capable of providing controlled lag time prior to drug release through a pressure-driven rupture mechanism. The invention provides a container-based dosage form configured to remain intact for a predetermined period following exposure to an aqueous environment and subsequently release one or more active pharmaceutical ingredients following rupture of the container structure. The lag time preceding release can be controlled through structural and geometric parameters of the system, including but not limited to ingress opening size, number, spatial distribution, and other design features influencing fluid ingress into the container.

The examples, applications, descriptions and content disclosed herein are exemplary and explanatory, and are non-limiting and non-restrictive in any way.

All scientific terms used herein have the same meaning as commonly used and understood by one of ordinary skill in the art. Examples, materials, methods, figures and tables are illustrative only and not intended to be limiting.

### Overall System Architecture

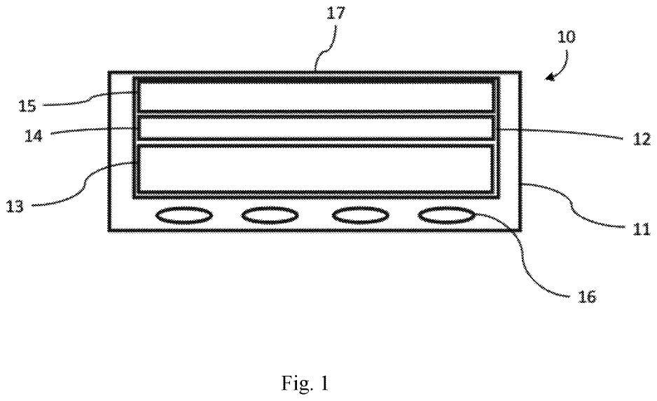

Referring to FIG. 1, an embodiment of a programmed delayed release container system 10 is illustrated in cross-section.

The container system 10 comprises a container shell 11 defining an internal cavity 12 configured to house internal compositions. Positioned within the internal cavity 12 are a swellable composition 13, an active pharmaceutical ingredient layer 15, and optionally a separating layer 14 located between the swellable composition and the active pharmaceutical ingredient layer.

One or more ingress openings 16 extend through the container shell 11 and provide controlled fluid communication between the external environment and the internal cavity 12.

In operation, external fluid enters the internal cavity 12 through the ingress openings 16 and contacts the swellable composition 13. Hydration of the swellable composition causes volumetric expansion within the confined internal cavity, generating internal pressure against the surrounding shell 11. When the internal pressure exceeds the mechanical strength of a portion of the shell 11, rupture occurs and the active pharmaceutical ingredient 15 is released.

The container system can comprise two or more structural components joined together. For example, the container shell can comprise two or more sections. Such joining can be accomplished by heat sealing, adhesive bonding, mechanical interlocking, threaded engagement, or snap-fit closure.

### Container Shell

The container shell 11 forms the structural enclosure of the container system and defines the boundaries of the internal cavity 12. The shell maintains the enclosure of the internal components during the lag phase preceding activation of the rupture mechanism.

In certain embodiments, the shell 11 is substantially impermeable to the passage of external fluids such that fluid ingress into the internal cavity occurs primarily through one or more ingress openings 16 formed in the shell. In this configuration, the shell serves as a barrier that prevents uncontrolled penetration of fluids into the system while permitting controlled fluid entry through the defined openings.

The shell material is preferably compatible with physiological environments and does not adversely interact with the active pharmaceutical ingredient, the swellable composition, or other components present within the dosage form.

In certain embodiments, the shell 11 comprises a polymeric material that is substantially insoluble, non-swellable, and non-erodible in gastrointestinal fluids during the lag period preceding rupture. Suitable polymers include hydrophobic cellulose derivatives such as ethyl cellulose, cellulose acetate, cellulose acetate butyrate, cellulose propionate, cellulose triacetate, cellulose diacetate, and cellulose acetate trimellitate.

Additional suitable polymers include methacrylate polymers and copolymers such as poly(alkyl methacrylates), methacrylic acid copolymers, and commercially available polymethacrylate materials including Eudragit® RS, RL, and NE. Further suitable materials include polyvinyl polymers such as polyvinyl acetate, and copolymers thereof, as well as polycaprolactone, polyurethanes, polyamides, polyethylene derivatives, polypropylene derivatives, and other hydrophobic polymers known in the art.

In other embodiments, the shell 11 comprises polymers that undergo gradual erosion, swelling, or dissolution in physiological environments. Such polymers can include hydrophilic cellulose derivatives such as hydroxypropyl methylcellulose, hydroxypropyl cellulose, hydroxyethyl cellulose, and methyl cellulose; polyethylene oxide; polyacrylic acid polymers including carbomers; polyvinyl alcohol; polyvinylpyrrolidone; and natural polysaccharides.

In certain embodiments, the shell material comprises polymers exhibiting pH-dependent solubility, swellability or degradation behavior within different regions of the gastrointestinal tract. Representative materials include enteric polymers such as cellulose acetate phthalate, hydroxypropyl methylcellulose phthalate, hydroxypropyl methylcellulose acetate succinate, cellulose acetate trimellitate, and methacrylic acid copolymers including Eudragit® L and Eudragit® S.

The polymeric material forming the shell 11 can further comprise plasticizers, stabilizers, or processing aids to facilitate fabrication and to modify mechanical properties of the shell. Representative plasticizers include triethyl citrate, polyethylene glycol, glycerol derivatives, and other pharmaceutically acceptable plasticizers.

The shell can be fabricated using a variety of manufacturing techniques. In certain embodiments, the shell is fabricated using additive manufacturing techniques including fused deposition modeling, stereolithography, selective laser sintering, or drop-on-demand printing. In other embodiments, the shell can be produced using injection molding, compression molding, capsule-forming processes, or other polymer processing techniques known in the pharmaceutical and polymer engineering arts

### Internal Cavity

The internal cavity 12 is defined by the surrounding container shell 11 and provides a confined environment for housing internal components of the dosage form. The internal cavity is sized and configured to accommodate the swellable composition 13 and the active pharmaceutical ingredient layer 15 positioned within the container system. In certain embodiments, the internal cavity further comprises a separating layer 14 positioned between the swellable composition and the active pharmaceutical ingredient layer to maintain spatial separation prior to activation of the system.

The internal cavity 12 is substantially isolated from the external environment except through the ingress openings 16, which regulate the entry of external fluid into the cavity.

The dimensions and geometry of the internal cavity can vary depending on the desired dosage form configuration. In certain embodiments, the internal cavity has a cylindrical geometry. However, other geometries including spherical, oval, capsule-shaped, or other three-dimensional configurations may also be employed.

The internal cavity provides a confined environment in which expansion of the swellable composition occurs following fluid ingress. As the swellable composition absorbs fluid and expands within the cavity, internal pressure develops against the surrounding shell structure. This pressure generation contributes to the rupture mechanism that ultimately results in release of the active pharmaceutical ingredient from the container system.

### Swellable Composition

The swellable composition 13 is positioned within the internal cavity 12 and functions as a pressure-generating component of the container system 10. The swellable composition is capable of absorbing fluid entering the cavity through the ingress openings 16 and expanding within the confined space of the internal cavity.

Upon contact with external fluid, the swellable composition 13 hydrates and undergoes volumetric expansion. Because the swellable composition is confined within the internal cavity 12 of the container shell 11, such expansion generates internal pressure against the surrounding shell structure. Continued swelling of the composition increases the internal pressure within the system until the mechanical strength of the shell is exceeded, thereby resulting in rupture of the container and release of the active pharmaceutical ingredient.

The swellable composition 13 may comprise one or more swellable polymers capable of absorbing fluid and expanding under physiological conditions. In many embodiments, the swellable composition forms a hydrated gel or hydrogel structure upon exposure to aqueous fluids, enabling volumetric expansion and pressure generation within the confined internal cavity.

Representative swellable polymers include hydroxypropyl methylcellulose, hydroxypropyl cellulose, hydroxyethyl cellulose, polyethylene oxide, sodium carboxymethyl cellulose, cross-linked polyacrylic acid polymers, sodium alginate, and other pharmaceutically acceptable swellable polymers known in the art.

In certain embodiments, the swellable composition comprises a cellulose-derived polymer selected from hydroxypropyl methylcellulose of varying molecular weights, including grades commonly referred to as K15M, K100M, and K200M. Such polymers are capable of forming hydrated gel structures upon exposure to aqueous environments and can generate sufficient swelling force when confined within the internal cavity 12 of the container system.

In certain embodiments, the swellable composition 13 comprises pH-independent hydrogels capable of swelling throughout a broad physiological pH range. Representative pH-independent swellable polymers include hydroxypropyl methylcellulose, hydroxypropyl cellulose, hydroxyethyl cellulose, polyethylene oxide, polyvinyl alcohol, polyvinylpyrrolidone, and other hydrophilic polymers capable of forming hydrated gel networks upon fluid absorption.

In other embodiments, the swellable composition 13 comprises pH-responsive or pH-dependent hydrogel polymers with swelling behavior that varies depending on the pH of the surrounding environment. Such polymers may remain substantially unswollen in acidic gastric environments while undergoing hydration and expansion under neutral or alkaline intestinal conditions. Representative pH-responsive polymers include cross-linked polyacrylic acid polymers, polymethacrylate-based polymers, carbomer polymers, poly(methacrylic acid) derivatives, and other ionizable polymer systems capable of undergoing pH-dependent swelling.

In certain embodiments, selection of pH-responsive swelling polymers enables the delayed-release container system to remain substantially inactive within the stomach and initiate swelling only after transit into intestinal regions of the gastrointestinal tract. Such configurations allow activation of the pressure-generating swelling mechanism within specific physiological environments, thereby enabling targeted rupture timing and site-specific drug release.

The swellable composition 13 may further comprise excipients or additives that modify swelling behavior, hydration rate, mechanical properties, or processing characteristics. Such additives include fillers, binders, plasticizers, lubricants, osmotic agents, pore-forming agents, or other pharmaceutically acceptable excipients.

The swellable behavior of the composition can be influenced by polymer molecular weight, crosslinking density, particle size, ionic character, hydrophilicity, and formulation composition. Selection of appropriate swellable materials and formulation parameters enables the swellable composition to generate sufficient pressure within the confined internal cavity to initiate rupture of the container shell 11 following fluid ingress.

In alternative embodiments, internal pressure within the cavity may be generated by mechanisms other than polymer swelling, including osmotic pressure generation or gas generation.

### Separating Layer

The delayed-release container system 10 may further comprise a separating layer 14 positioned within the internal cavity 12 between the swellable composition 13 and the active pharmaceutical ingredient layer 15. The separating layer functions to maintain spatial separation between the swellable composition and the drug-containing portion of the system. The separating layer 14 serves to prevent premature interaction between the swellable composition 13 and the active pharmaceutical ingredient layer 15. In this embodiment, the separation acts to preserve the independency of the swellable composition and the drug layer. This separation allows for the drug layer to exhibit release behavior unaffected by the swelling composition, such as rapid pulsatile release.

The separating layer 14 may comprise one or more pharmaceutically acceptable polymers capable of forming a structurally stable barrier within the internal cavity. Representative materials suitable for forming the separating layer include ethyl cellulose, cellulose acetate, polycaprolactone, polymethacrylate, other pharmaceutical polymers known in the art that can act as an inert unreactive barrier and combinations thereof.

In alternative embodiments, the separating layer 14 may be omitted entirely, such that the swellable composition 13 is positioned directly in contact with the active pharmaceutical ingredient layer 15 within the internal cavity 12. In such embodiments, the swellable composition may interact and directly transmit pressure to the drug-containing portion of the dosage form upon hydration and expansion. In this embodiment, the interaction may alter the release behavior where the drug layer via the swellable composition containing release modifiers such as pharmaceutical polymers. This interaction may result in sustained release behavior rather than rapid and pulse release.

The thickness, composition, mechanical strength, structural properties, and composition of the separating layer 14 can be adjusted to achieve desired structural characteristics of the container system. In certain embodiments, these properties may further be selected to influence the swellability and transmission of mechanical force generated by swelling of the swellable composition, thereby enabling modulation of pressure transfer within the internal cavity and adjustment of the lag time or release behavior of the dosage form.

### Active Pharmaceutical Ingredient Layer

The delayed-release container system 10 further comprises an active pharmaceutical ingredient layer 15 positioned within the internal cavity 12 of the container shell 11. The active pharmaceutical ingredient layer contains one or more therapeutic agents intended for release following rupture of the container system.

The active pharmaceutical ingredient layer 15 may comprise a pharmaceutically effective amount of an active pharmaceutical ingredient combined with pharmaceutically acceptable excipients. Such excipients may include binders, fillers, disintegrants, lubricants, stabilizers, or other materials commonly used in pharmaceutical solid dosage forms.

The active pharmaceutical ingredient may be selected from a wide range of therapeutic agents suitable for delayed or chronotherapeutic delivery. Representative classes of active pharmaceutical ingredients include corticosteroids, antihypertensive agents, analgesics, anti-inflammatory agents, antibiotics, antidiabetic agents, and other therapeutic compounds where timed release may provide therapeutic benefit.

The active pharmaceutical ingredient may comprise small-molecule drugs, peptides, proteins, nucleic acids, or other biologically active agents suitable for therapeutic administration. In certain embodiments, the active pharmaceutical ingredient may be present as a conventional small-molecule compound, while in other embodiments the active pharmaceutical ingredient may comprise a larger biomolecule or biologic formulation optionally incorporated within a carrier system such as microparticles, nanoparticles, lipid-based carriers, polymeric matrices, or other delivery vehicles.

In certain embodiments, the active pharmaceutical ingredient comprises hydrocortisone or a pharmaceutically acceptable salt or derivative thereof. Hydrocortisone is particularly suitable for delayed-release administration in the treatment of disorders such as adrenal insufficiency, where synchronization of drug release with circadian physiological rhythms may improve therapeutic outcomes.

The active pharmaceutical ingredient layer 15 may be formulated in the form of a compressed powder, compacted tablet, granulated formulation, multiparticulate system, pellets, microgranules, or other solid dosage forms suitable for positioning within the internal cavity 12 of the container system. In certain embodiments, the active pharmaceutical ingredient may be present as a loose powder, compacted mass, pelletized particles, coated granules, or other solid structures capable of being retained within the internal cavity prior to activation of the rupture mechanism.

Upon rupture of the container shell 11, the active pharmaceutical ingredient layer 15 is expelled or exposed to the surrounding environment, thereby enabling rapid release, dissolution, or dispersion of the therapeutic agent into the surrounding fluid. In certain embodiments, the active pharmaceutical ingredient layer may further comprise disintegrating excipients configured to promote rapid disintegration and dissolution of the drug-containing portion following rupture of the container system.

### Ingress Openings

The container shell 11 further comprises one or more ingress openings 16 formed in the shell structure. These ingress openings permit penetration of external fluid into the internal cavity 12 of the container system.

The ingress openings 16 function as controlled fluid-entry points that initiate hydration of the swellable composition 13 positioned within the internal cavity. Upon administration of the dosage form and exposure to physiological fluids, external fluid enters the cavity through the ingress openings and contacts the swellable composition.

The number, size, shape, and spatial distribution of the ingress openings 16 may be adjusted to regulate the rate at which external fluid enters the internal cavity 12. By controlling the rate of fluid ingress, the hydration rate of the swellable composition may be modulated, thereby influencing the rate of swelling and the time required to generate sufficient internal pressure to rupture the container shell 11.

In certain embodiments, the ingress openings 16 comprise perforations extending through a portion of the container shell. The perforations may be circular, elliptical, polygonal, slot-shaped, or otherwise shaped openings that allow controlled fluid entry into the internal cavity.

The dimensions of the ingress openings 16 may be selected to permit penetration of physiological fluids while substantially retaining the swellable composition 13 within the internal cavity 12 during the lag period preceding rupture. If the openings become excessively large, portions of the swellable composition may escape through the openings, thereby relieving internal pressure and preventing rupture of the container structure. Conversely, if the openings are sufficiently small, the rate of fluid ingress may be reduced, thereby extending the lag time preceding rupture.

Adjustment of perforation size provides a mechanism for programming the lag time of the delayed-release container system. Larger perforations permit faster fluid ingress and more rapid hydration of the swellable composition, resulting in shorter lag times prior to rupture. Conversely, smaller perforations restrict fluid ingress and prolong the time required for swelling and pressure buildup, thereby extending the lag time prior to drug release.

The ingress openings 16 can be formed using a variety of manufacturing techniques, including mechanical drilling, laser perforation, molding processes, or additive manufacturing techniques used to fabricate the container shell 11.

By controlling the structural parameters of the ingress openings, the delayed-release container system provides a tunable mechanism for adjusting the lag time prior to drug release.

### Mechanism of Operation

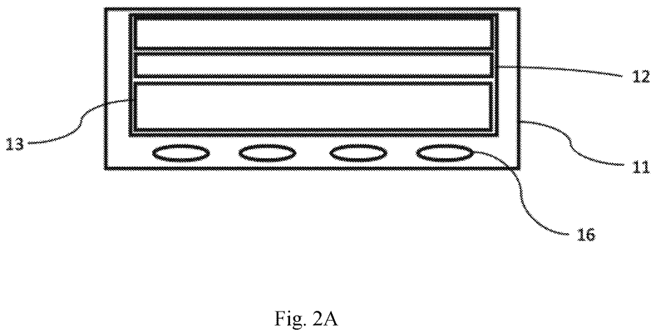

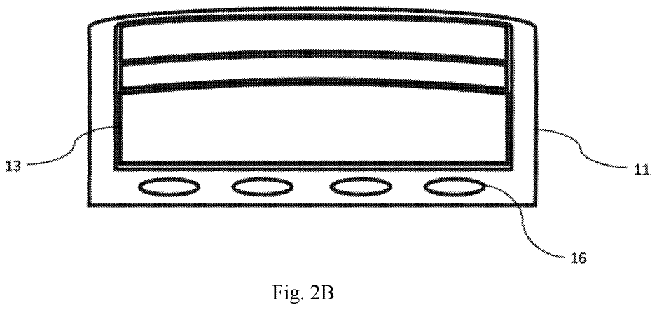

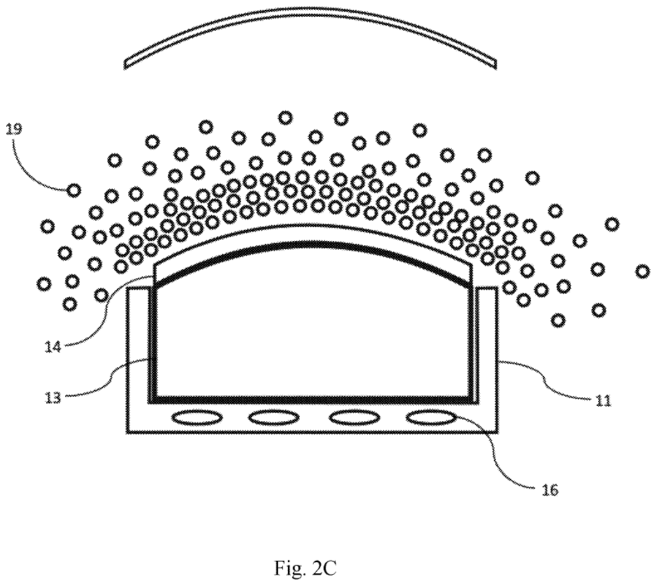

The operational mechanism of the container system 10 is illustrated herein (FIGS. 2A-2C). In FIG. 2A, the system is shown in its initial intact state prior to activation. The container shell 11 encloses the internal cavity 12, which contains the swellable composition 13 and the active pharmaceutical ingredient layer 15, optionally separated by the separating layer 14.

Following exposure to an aqueous environment, such as gastrointestinal fluid, external fluid enters the system through the ingress openings 16 and contacts the swellable composition 13.

In FIG. 2B, the swellable composition 13 begins to hydrate and expand as it absorbs the incoming fluid. Because expansion occurs within the confined internal cavity 12, internal mechanical pressure is generated against the surrounding container shell 11.

As swelling continues, internal pressure within the container progressively increases until it exceeds the mechanical strength of a portion of the container structure.

In FIG. 2C, rupture of the container shell 11 occurs, permitting rapid release of the active pharmaceutical ingredient layer 15 into the surrounding environment. In some embodiments, rupture may occur at a cap or closure region of the container. In other embodiments, rupture may occur at other structurally defined locations depending on device design and material properties.

The rupture event results in rapid expulsion or exposure of the internal drug composition and initiates drug release after the programmed lag phase.

### Programmable Lag Time Through Structural Design

A key feature of the present invention is the ability to control the lag time preceding drug release through modification of structural parameters of the system.

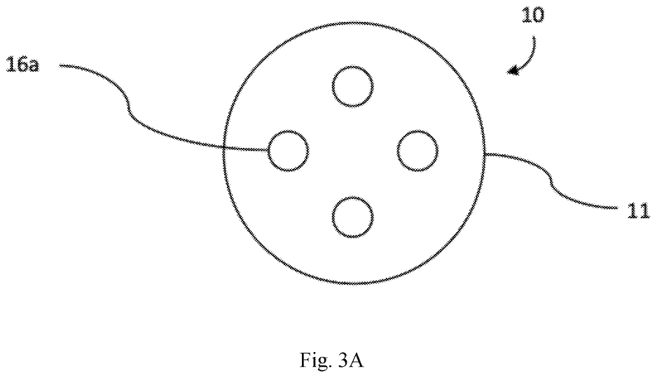

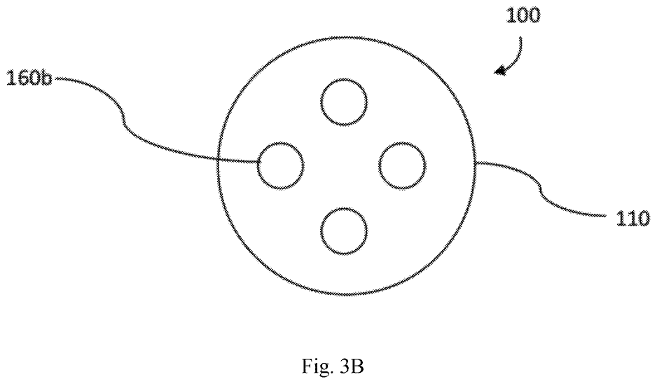

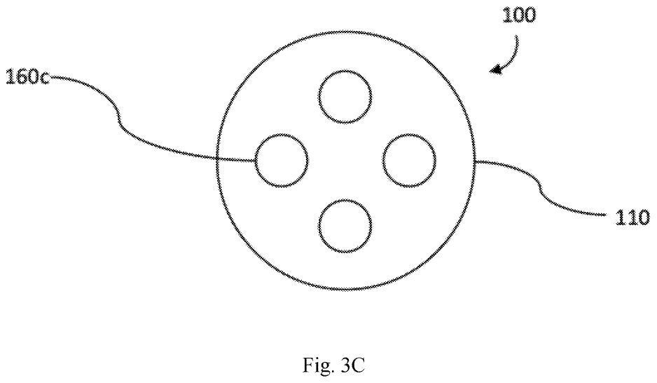

Different embodiments of the container system are illustrated comprising ingress openings 16 of different sizes (FIGS. 3A-3C). In FIG. 3A, relatively small ingress openings 16a are provided. In FIG. 3B, medium-sized ingress openings 16b are provided. In FIG. 3C, relatively large ingress openings 17c are provided.

Smaller openings permit slower fluid ingress into the system and may result in longer lag times prior to rupture. Intermediate openings may produce intermediate lag times. Larger openings permit faster fluid ingress and shorter lag times prior to rupture.

Accordingly, the lag time prior to rupture may be tuned by modifying one or more structural parameters of the device, including the size, number, geometry, spatial distribution, or placement of ingress openings 16, the thickness and mechanical properties of the shell 11, and the composition and swelling characteristics of the swellable composition 13.

Through such design modifications, the system may be programmed to achieve desired delay intervals prior to drug release.

### Experimental Characterization and Representative Performance Data

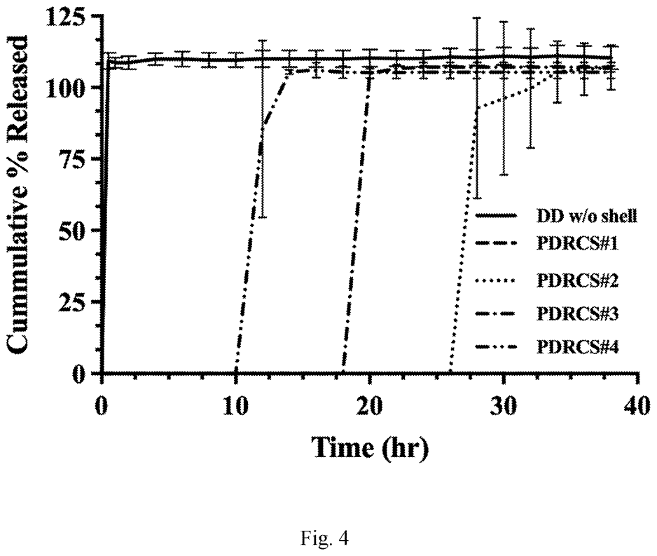

Representative drug release profiles are shown herein (FIG. 4) illustrating the lag-time behavior of the delayed-release container system. The release profiles demonstrate that modification of system design parameters, particularly perforation size, produces different delay periods prior to release of the active pharmaceutical ingredient.

Release from the present system is governed primarily by controlled fluid ingress and pressure-driven rupture rather than by diffusion of the active pharmaceutical ingredient through the shell.

### Materials and Manufacturing

The container system 10 may be fabricated using various pharmaceutical manufacturing methods. In certain embodiments, the container shell 11 may be produced using additive manufacturing techniques, including fused deposition modeling, stereolithography, selective laser sintering, drop-on-demand printing, or other three-dimensional printing processes.

In other embodiments, conventional manufacturing techniques such as injection molding, compression molding, capsule-forming processes, heat-sealing processes, or other polymer processing techniques may be used.

The internal components, including the swellable composition 13, separating layer 14, and active pharmaceutical ingredient layer 15, may be fabricated using compression, granulation, molding, casting, lyophilization, particulate filling, or other suitable pharmaceutical processing methods.

### Applications

The programmable delayed-release container system may be useful in various therapeutic applications where delayed drug release is desirable. Representative applications include chronotherapeutic drug delivery, time-dependent dosing regimens, and therapies requiring drug release following transit through specific regions of the gastrointestinal tract.

Because the lag time preceding release may be programmed through structural design, the system allows flexible customization of drug release profiles for particular therapeutic needs.

### Variations and Modifications

While specific embodiments of the invention have been illustrated and described herein, the invention is not limited to the particular embodiments described.

Various modifications, substitutions, and alterations may be made without departing from the spirit and scope of the invention. For example, the shape and dimensions of the container shell 11, the location and geometry of ingress openings 16, the composition of the swellable material 13, the presence or absence of the separating layer 14, and the arrangement of internal components within the cavity 12 may be modified depending on design requirements.

Similarly, different manufacturing techniques, materials, and active pharmaceutical ingredients may be employed while still achieving the pressure-driven delayed-release mechanism described herein.

In contrast to many conventional delayed-release oral dosage forms that rely primarily on diffusion-controlled release, erosion-controlled release, or pH-triggered coating dissolution, the present invention utilizes a pressure-driven rupture mechanism in which drug release occurs only after sufficient internal pressure has been generated within the confined cavity of the system. In such embodiments, the container shell substantially prevents diffusion of the active pharmaceutical ingredient through the shell prior to rupture, thereby providing a true lag phase during which little or no drug release occurs before the rupture event.

Accordingly, the invention encompasses all modifications, equivalents, and alternative configurations that fall within the scope of the appended claims.

### Example 1—Preparation of Ethyl Cellulose Filament for Fabrication of the Container Shell

A filament suitable for fabrication of a delayed-release container shell was prepared by hot-melt extrusion.

Ethyl cellulose, triethyl citrate, and hydroxypropyl cellulose were blended in a weight ratio of 80:10:10. The powder blend was mixed for approximately 10 minutes and then processed using a twin-screw hot-melt extruder.

The extrusion temperature was maintained at 130 °C, except for the first two zones which were maintained at 25 °C. The screw speed was 50 rpm and the feed rate was 2.1±0.1 g/min.

During extrusion, torque ranged from 4.2 to 4.8 Nm, and die pressure was approximately 13 bar. A cylindrical die having a diameter of 2.5 mm was used, producing filaments having a diameter of 2.4 ±0.05 mm.

The resulting filament exhibited sufficient uniformity and mechanical integrity for use in fabrication of container shells for delayed-release dosage forms.

### Example 2—Preparation of Internal Components

Internal components of the delayed-release container system were prepared as compressed discs.

A swelling disc was prepared using hydroxypropyl methylcellulose (HPMC K200M), with each disc containing 50 mg of HPMC.

A separating disc was prepared using ethyl cellulose, with each disc containing 20 mg of ethyl cellulose.

A drug disc was prepared using a formulation containing:
mannitol;
hydrocortisone;
sodium starch glycolate; and
silicified microcrystalline cellulose

Each component was present at 10 mg, corresponding to 25 wt. % of the drug disc composition.

The swelling disc was compressed using 8 mm flat-faced tooling at 5000 psi. The separating ethyl cellulose layer was then placed over the swelling disc and compressed under the same pressure to form a bilayer structure.

The drug disc was compressed separately using 2000 psi.

### Example 3—Fabrication and Assembly of the Programmable Delayed-Release Container System

A digital model of the outer shell was prepared and converted into machine-readable printing instructions.

The container shell was fabricated using fused deposition modeling (FDM) with the ethyl cellulose-based filament prepared in Example 1.

Printing parameters included:
layer height: 0.1 mm
nozzle temperature: 190 °C
bed temperature: 45 °C
print speed: 6 mm/s

The printing instructions included a programmed pause prior to formation of the top cap.

At the pause stage, the pre-compressed internal discs prepared in Example 2 were manually inserted into the partially printed shell. Printing was then resumed to seal the device.

In one embodiment, the shell had the following dimensions:
shell height: 3.0 mm
shell width: 10.7 mm
shell wall thickness: 0.8 mm
shell bottom height: 1.0 mm
top cap thickness: 0.2 mm

The internal layers had the following dimensions:
swelling layer height: 0.8 mm, width 8 mm
separating layer height: 0.4 mm, width 8 mm
drug layer height: 0.6 mm, width 8 mm

Four ingress perforations were formed in the base of the shell. Each perforation extended through shell thickness.

Devices were fabricated with perforation diameters of:
mm
1.5 mm
2.0 mm

The perforations were positioned symmetrically on the base of the container.

### Example 4—Swelling Behavior of the Internal Hydrogel Disc

The swelling behavior of the HPMC swelling disc was evaluated in media representing physiological pH environments.

Individual discs were immersed in 250 mL of the following media:
M hydrochloric acid (pH 1.2)
phosphate buffer (pH 6.8)
Phosphate Buffer (pH 7.4)

Disc swelling was monitored by measuring the weight of the discs at 30-minute intervals for a period of 3 hours.

Swelling profiles obtained in the different media were substantially similar, indicating that the swelling behavior of the HPMC disc was largely independent of pH over the tested range.

### Example 5—Mechanical Rupture Characterization

Mechanical strength of the container top cap was evaluated using a texture analyzer equipped with a cylindrical stainless-steel probe.

A custom holder was used to position the container shell vertically during testing. Compression was applied at a speed of 0.5 mm/s with a 0.05 N trigger force.

The break force was recorded as the maximum force immediately prior to rupture of the cap.

Across three replicates, the average break force of the cap was 2.76±0.03 N.

Scanning electron microscopy of sectioned shells revealed that the interface between the cap and the shell wall exhibited reduced interlayer bonding relative to the wall-to-wall printed layers. This region corresponded to the programmed pause introduced during the printing process and formed a preferential rupture site.

### Example 6—In Vitro Delayed Release and Effect of Perforation Diameter

Three versions of the delayed-release container system were evaluated in vitro. The devices differed only in perforation diameter:
mm
1.5 mm
2.0 Mm

Dissolution testing was conducted in 900 mL distilled water using USP Apparatus II at 100 rpm and 37.5±0.5 °C

Hydrocortisone release was quantified spectrophotometrically at 248 nm.

Devices with 1.0 mm perforations exhibited rupture and drug release at approximately 28 hours. Devices with 1.5 mm perforations exhibited rupture and release at approximately 20 hours. Devices with 2.0 mm perforations exhibited rupture and release at approximately 12 hours.

A drug disc tested without the enclosing shell exhibited immediate release. A shell without perforations did not rupture during the experimental period.

These observations demonstrate that the size of the ingress openings influences the lag time preceding rupture of the container system.

### Example 7—Empirical Relationship Between Perforation Diameter and Rupture Time

The relationship between perforation diameter and rupture lag time was described using the following empirical regression model:

t=30.86/d−2.29
where:
t=rupture time (hours)
d=Perforation Diameter (millimeters)

The model predicted decreasing rupture time with increasing perforation diameter, consistent with the experimental observations described in Example 6.

This relationship demonstrates that perforation diameter can serve as a tunable geometric parameter for programming delayed drug release.

### Example 8—Alternative Swellable Polymer Embodiment

In another embodiment, the swellable composition may comprise a polymer different from the hydroxypropyl methylcellulose described in Example 2.

For example, a swelling layer may be prepared using high-molecular-weight polyethylene oxide (PEO) having a molecular weight between approximately 4,000,000 and 7,000,000 Daltons. The polyethylene oxide powder may be compressed into a disc using compression tooling similar to that described in Example 2.

The polyethylene oxide swelling disc may be positioned within the internal cavity of the container shell in a configuration similar to that described in Example 3, optionally together with a separating layer and a drug-containing layer.

Upon exposure to aqueous media entering the container through the ingress openings, the polyethylene oxide may hydrate and undergo volumetric expansion within the confined cavity. Hydration of the polyethylene oxide is expected to generate internal pressure as the polymer swells, which can increase until the pressure exceeds the mechanical strength of the container shell, thereby causing rupture of the container and release of the active pharmaceutical ingredient.

This embodiment illustrates that swellable compositions suitable for use in the delayed-release container system may include polymers other than hydroxypropyl methylcellulose. Representative swellable polymers include polyethylene oxide, poly(acrylic acid) polymers, carbomer polymers, sodium alginate, cross-linked polyacrylate materials, cellulose derivatives, and other pharmaceutically acceptable hydrophilic polymers capable of absorbing fluid and expanding within a confined cavity to generate pressure.

### Example 9—Alternative Container Manufacturing Method

In another embodiment, the container shell of the delayed-release system may be fabricated using manufacturing methods other than additive manufacturing.

For example, a container shell may be produced using injection molding of a pharmaceutical polymer composition such as ethyl cellulose or other suitable polymeric materials. In such an embodiment, the molded shell may define a hollow cavity configured to receive the swelling layer and the active pharmaceutical ingredient layer.

The swelling composition and drug-containing components may be inserted into the cavity of the molded shell, after which the container may be sealed using a closure element such as a press-fit cap, heat-sealed polymer film, solvent-bonded lid, or other suitable sealing mechanism.

Ingress openings may be formed in the shell using mechanical drilling, laser perforation, or molding features incorporated into the mold design.

Following assembly, the container system may operate in a manner similar to the embodiments described in the preceding examples. Fluid entering the container through the ingress openings hydrates the swellable composition, resulting in expansion and generation of internal pressure within the confined cavity. As pressure increases, rupture of the container structure may occur, thereby releasing the active pharmaceutical ingredient after a programmed delay period.

This example demonstrates that the delayed-release container system may be fabricated using a variety of pharmaceutical manufacturing techniques, including injection molding, compression molding, capsule-forming processes, thermoforming, casting, or other polymer processing methods.

These examples demonstrate that delayed-release container systems according to the present invention can be reproducibly fabricated and can provide programmable lag times prior to drug release through structural design of the ingress openings and internal swelling mechanism.

Now, therefore, the following is claimed:

1. A delayed-release oral dosage composition comprising:
a container structure defining an internal cavity;
a swellable composition contained within the internal cavity;
an active pharmaceutical ingredient contained within the container structure; and
one or more ingress openings in the container structure;
wherein the ingress openings are configured to permit entry of external fluid into the internal cavity; and
wherein the swellable composition is configured to absorb external fluid and expand within the internal cavity to generate internal pressure until rupture of at least a portion of the container structure occurs to effect release of the active pharmaceutical ingredient.

2. The composition of claim 1, wherein the container structure has a cylindrical geometry.

3. The composition of claim 1, wherein the container structure has a spherical geometry.

4. The dosage form of claim 1, wherein the container structure has a capsule-shaped geometry.

5. The composition of claim 1, wherein the container structure comprises a pharmaceutically acceptable polymer.

6. The composition of claim 1, wherein the ingress openings are positioned in a lateral surface of the container structure.

7. The composition of claim 1, wherein the ingress openings are positioned on a top surface of the container structure.

8. The composition of claim 1, wherein the ingress openings are positioned on a bottom surface of the container structure.

9. The composition of claim 1, wherein the internal cavity comprises a single compartment.

10. The composition of claim 1, wherein the internal cavity comprises a plurality of compartments.

11. The composition of claim 10, wherein a distinct active pharmaceutical ingredient is contained with each of at least two compartments.

12. The composition of claim 10, wherein the compartments are configured to produce different lag times preceding release of the active pharmaceutical ingredient in each compartant upon exposure of the composition to an external fluid.

13. The composition of claim 1, wherein the swellable composition comprises a swellable polymer.

14. The composition of claim 1 further comprising a separating layer positioned between the swellable composition and the active pharmaceutical ingredient.

15. The composition of claim 13, wherein the separating layer comprises a pharmaceutically acceptable polymer.

16. The composition of claim 1, wherein the swellable composition directly contacts the active pharmaceutical ingredient.

17. The composition of claim 1, wherein the active pharmaceutical ingredient comprises a small molecule drug.

18. The composition of claim 1, wherein the active pharmaceutical ingredient comprises at least one peptide and/or protein.

19. The composition of claim 1, wherein the active pharmaceutical ingredient is formulated in the form of a compressed powder.

20. A method of delayed delivery of an active pharmaceutical ingredient to a subject comprising the step of administering to a subject a delayed-release oral dosage form comprising: a container structure defining an internal cavity; a swellable composition contained within the internal cavity; an active pharmaceutical ingredient contained within the container structure; and one or more ingress openings in the container structure; wherein the ingress openings are configured to permit entry of external fluid into the internal cavity.
